(19) United States
(12) Patent Application Publication
(10) Pub. No.: US 2022/0331517 A1
(43) Pub. Date: Oct. 20, 2022

(54) SYRINGE PUMP CONTROLLER

(71) Applicants: The Trustees of Indiana University, Bloomington, IN (US); The United States Government as represented by the Department of Veterans Affairs, Washington D.C., DC (US); Purdue Research Foundation, West Lafayette, IN (US)

(72) Inventors: Mark S. Carlsen, Lafayette, IN (US); Gregory S. Eakins, West Lafayette, IN (US); Robert M. Everly, West Lafayette, IN (US); Catherine L. McIntyre, West Lafayette, IN (US); Phillip J. Wyss, West Lafayette, IN (US); Robert L. Bacallao, Indianapolis, IN (US)

(73) Assignees: The Trustees of Indiana University, Bloomington, IN (US); The United States Government as represented by the Department of Veterans Affairs, Washington D.C., DC (US)

(21) Appl. No.: 17/760,657

(22) PCT Filed: Sep. 17, 2020
(86) PCT No.: PCT/US2020/051161

§ 371 Date: Mar. 15, 2022

Related U.S. Application Data
(60) Provisional application No. 62/901,437, filed on Sep. 17, 2019.

(51) Int. Cl. A61M 5/168; A61M 5/145

(52) U.S. Cl. CPC A61M 5/16854; A61M 5/14546; A61M 2210/1082

(57) ABSTRACT

Syringe pump controllers which memorialize the process of hydrodynamic isotonic saline delivery (HIFD) to reverse acute kidney injury are disclosed. In some embodiments, the syringe pump controllers are constructed to control syringe pump(s) to deliver a defined volume of saline at a prescribed perfusion rate while monitoring renal vein pressures during the infusion. In some embodiments, the syringe pump controllers have safety features designed to shut off the infusion if the renal vein pressure goes above set safety limits.

### CROSS-REFERENCE TO RELATED APPLICATION

The present application claims benefit of U.S. Provisional Application No. 62/901,437, filed on Sep. 17, 2019, titled “SYRINGE PUMP CONTROLLER” which is herein incorporated by reference in its entirety.

### FIELD OF THE DISCLOSURE

This disclosure relates generally to fluid delivery, and more specifically to fluid delivery control system for treating kidney injury.

### BACKGROUND OF THE DISCLOSURE

Kidneys are susceptible to ischemia-reperfusion (FR) injury which can originate from myocardial infarction, renal trauma, and transplant. As such, there is a need for an effective method to reverse such acute kidney injuries.

### SUMMARY OF THE DISCLOSURE

Various embodiments of the present disclosure provide syringe pump controllers which memorialize the process of hydrodynamic isotonic saline delivery (HIFD) to reverse acute kidney injury. This is achieved by performing intravenous fluid delivery to administer saline solution to be delivered to the damaged kidney in order to restore the original effectiveness of the kidney. In some embodiments, the syringe pump controllers are constructed to control syringe pump(s) to deliver a defined volume of saline at a prescribed perfusion rate while monitoring renal vein pressures during the infusion. In some embodiments, the syringe pump controllers have safety features designed to shut off the infusion if the renal vein pressure goes above set safety limits.

Some embodiments provide a syringe pump controller having a processing unit. The processing unit includes at least one microcontroller and a receiver. The microcontroller is operatively coupled to a syringe pump and the receiver operatively coupled to a pressure sensor. The processing unit may receive pressure sensor data via the receiver, determine whether the pressure sensor data exceeds a predetermined setpoint pressure, and stop the syringe pump in response to determining that the pressure sensor data exceeds the setpoint pressure. In some examples, the syringe pump controller includes a catheter fluidly coupled with the syringe pump and configured to deliver fluid from the syringe pump.

In some examples, the pressure sensor is positioned parallel to the syringe pump. In some examples, the pressure sensor measures a pressure within the catheter located distally of an exit portion of the syringe pump. In some examples, the catheter includes a first end fluidly coupled with the exit portion of the syringe pump and a second end where fluid delivered from the syringe pump exits. In some examples, the pressure sensor is positioned at a body portion of the syringe pump. In some examples, the pressure sensor is positioned at the first end of the catheter. In some examples, the pressure sensor is positioned between the first end and the second end of the catheter. In some embodiments, the syringe pump controller includes a plurality of syringe pumps, each configured to deliver a predetermined fluid at a predetermined rate into the catheter. Each of the plurality of syringe pumps may have an exit portion fluidly coupled with the first end of the catheter. The processing unit may stop one or more of the plurality of syringe pumps in response to determining that the pressure sensor data in the catheter exceeds the setpoint pressure.

Some embodiments provide methods of operating or controlling a syringe pump. A processing unit coupled with the syringe pump may determine a pressure setpoint, activate the syringe pump, measure fluid pressure, determine whether the measured fluid pressure exceeds the pressure setpoint based on the fluid pressure, and in response to determining that the measured fluid pressure exceeds the pressure setpoint, automatically stop the syringe pump. In some examples, the pressure sensor is positioned at the syringe pump or a catheter extending from the syringe pump. In some examples, there may be a plurality of syringe pumps operated or controlled by the processing unit. In some examples, the processing unit stops one or more of the plurality of syringe pumps in response to determining that the pressure sensor data in the catheter exceeds the setpoint pressure.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

The embodiments will be more readily understood in view of the following description when accompanied by the below figures and wherein like reference numerals represent like elements. These depicted embodiments are to be understood as illustrative of the disclosure and not as limiting in any way.

FIG. 1 shows a schematic diagram of a syringe pump controller performing an HIFD procedure to a kidney of a patient according to one embodiment.

FIG. 2 shows a block diagram of the syringe pump controller and other hardware operatively coupled thereto according to one embodiment.

FIG. 3 shows an angled view of a syringe pump controller according to one embodiment.

FIG. 4 shows a table of pump program parameters according to one embodiment.

FIG. 5 shows a schematic diagram of a processing unit used in a syringe pump controller according to one embodiment.

FIG. 6 shows a flow diagram of a method of controlling the syringe pump as performed by a syringe pump controller according to one embodiment.

### DETAILED DESCRIPTION OF THE DISCLOSURE

In the following detailed description, reference is made to the accompanying drawings which form a part hereof, and in which is shown by way of illustration specific embodiments in which the present disclosure is practiced. These embodiments are described in sufficient detail to enable those skilled in the art to practice the present disclosure, and it is to be understood that other embodiments can be utilized and that structural changes can be made without departing from the scope of the present disclosure. Therefore, the following detailed description is not to be taken in a limiting sense, and the scope of the present disclosure is defined by the appended claims and their equivalents.

Reference throughout this specification to “one embodiment,” “an embodiment,” or similar language means that a particular feature, structure, or characteristic described in connection with the embodiment is included in at least one embodiment of the present disclosure. Appearances of the phrases “in one embodiment,” “in an embodiment,” and similar language throughout this specification may, but do not necessarily, all refer to the same embodiment. Similarly, the use of the term “implementation” means an implementation having a particular feature, structure, or characteristic described in connection with one or more embodiments of the present disclosure, however, absent an express correlation to indicate otherwise, an implementation may be associated with one or more embodiments. Furthermore, the described features, structures, or characteristics of the subject matter described herein may be combined in any suitable manner in one or more embodiments.

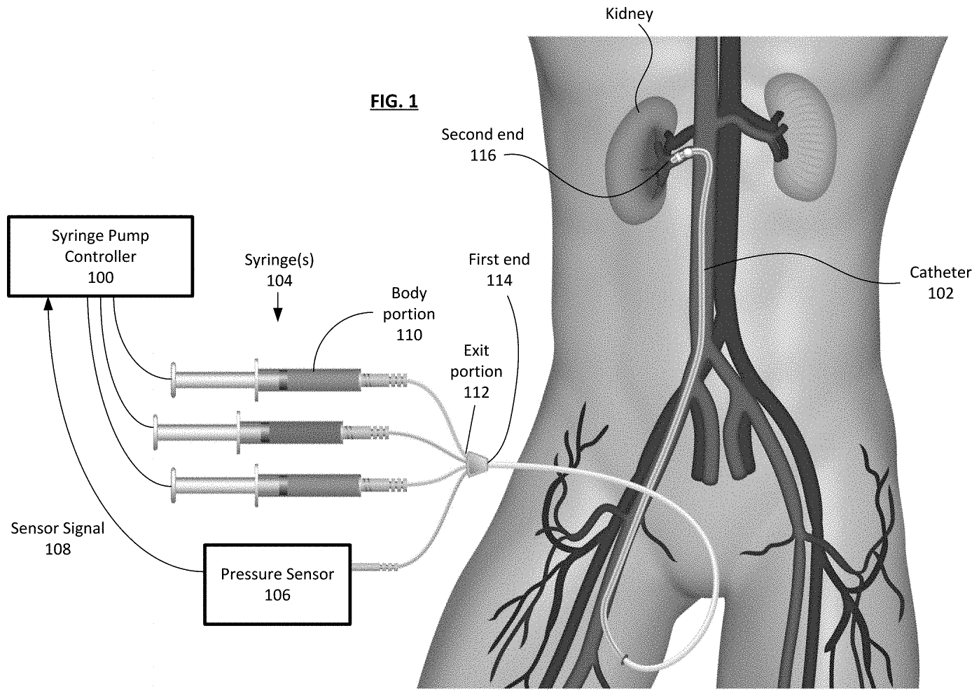

FIG. 1 shows a schematic diagram of a syringe pump controller 100 which delivers fluid such as saline solution to the kidney(s) of a patient via a catheter 102. At the other end of the catheter 102 is a syringe 104 (or a set of syringes wherein each syringe contains a different type of chemical solution) through which the fluid is delivered, and the catheter 102 is coupled with a pressure sensor 106 which measures the pressure level within the catheter 102 such that sensor signal 108 measured by the sensor 106 is then sent to the syringe pump controller 100 for analysis. The pressure sensor 106 may be positioned parallel to the syringe 104 so as not to interrupt the flow of fluid through the catheter 102, thereby ensuring steady flow of fluid through the catheter 102 while the syringe pump controller 100 is operating. In some embodiments, the location of the pressure sensor 106 may be anywhere along the entire length of the catheter 102.

The syringe 104 has a body portion 110 and an exit portion 112. The catheter 102 has a first end 114 and a second end 116. The first end of the catheter 102 is coupled with the exit portion 112 of the syringe 104, and the second end 116 is where the fluid delivered from the syringe 104 exits. In some examples, the fluid is delivered to the kidney of the patient. In some examples, the pressure sensor 106 may be positioned at the body portion 110, located distally of the exit portion 112, positioned at the first end 114, or positioned such that the pressure measurements are taken at the body portion 110 of the syringe 104 or at the exit portion 112 of the syringe 104. In some examples, the pressure sensor 106 may be positioned such that the pressure measurements are taken within the catheter 102 at a location between the first end 114 and the second end 116.

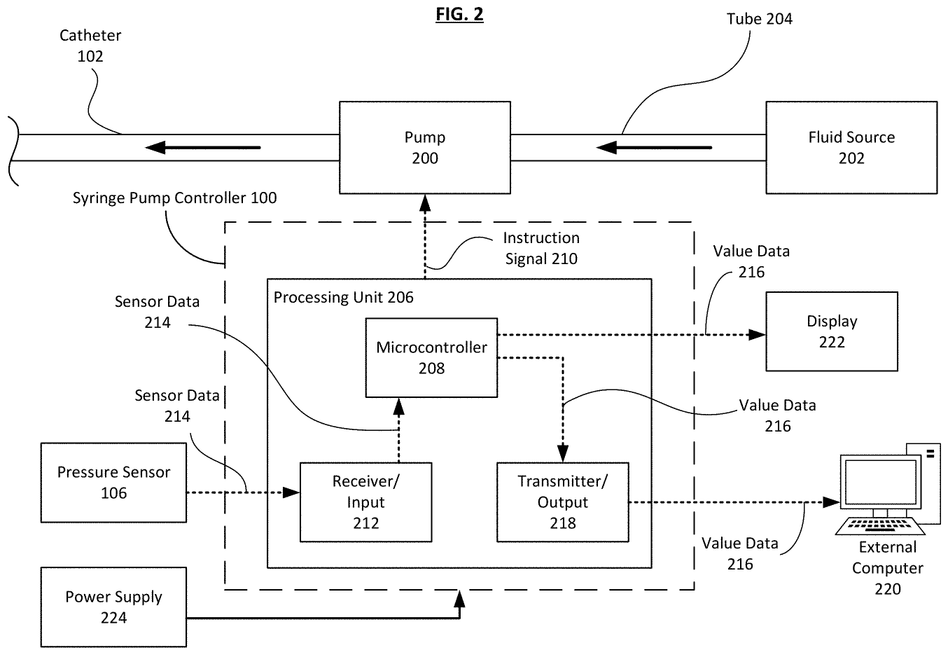

FIG. 2 shows a schematic diagram of the syringe pump controller 100 in detail. Specifically, the syringe pump controller 100 is operative coupled to an external pump 200, a fluid source 202 fluidly coupled with the pump 200 via a tube 204, and a processing unit 206 which receives and processes the sensor signal 108. Specifically, the processing unit 206 includes a microcontroller 208 that determines if the pump 200 needs to be stopped. If so, the processing unit 206 sends instruction signals 210 to the pump 200 to stop its operation. When there are a plurality of syringes or syringe pumps, each of them may be separately or individually stopped in response to the processing unit 206 determining that the pumps need to be stopped.

The processing unit 206 also includes a receiver or input 212 to receive sensor data 214 and transmits it to the microcontroller 208 for processing. The processing unit 206 in some examples also includes a transmitter or output device 218 which exports or outputs value data 216 as produced by the microcontroller 208 to an external computer 220. In some examples, the external computer 220 receives the data for further processing or data collection, such as for future prognosis or patient health monitoring purposes, among other applications known in the art. In some examples, an internal computer (not shown) may be implemented within the syringe pump controller 100 to perform the tasks associated with the external computer 220 as explained above. Additionally, the microcontroller 208 can transmit the value data 216 to a display 222 connected to the syringe pump device 100. Lastly, a power supply 224 is connected to and supplies power to the syringe pump device 100.

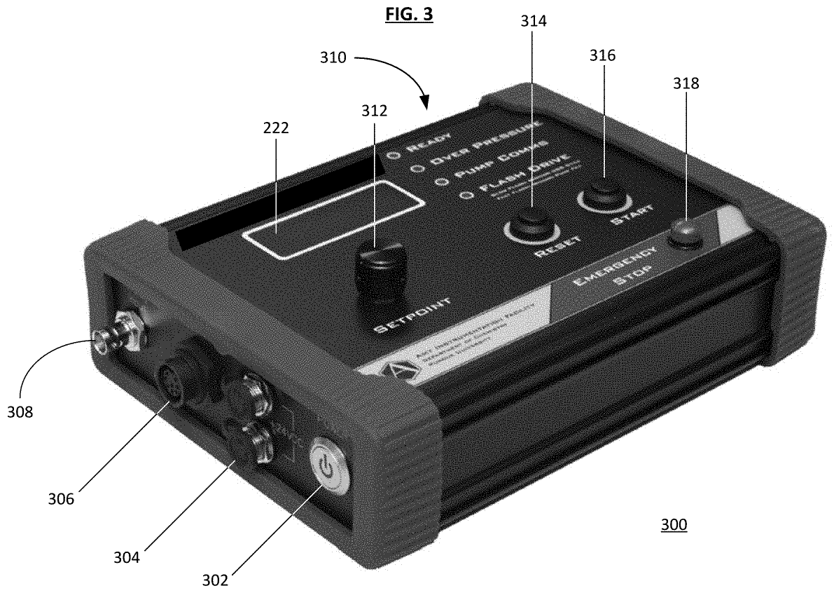

FIG. 3 shows an example of an exterior view of a syringe pump controller 300 according to one embodiment. The syringe pump controller 300 includes a power switch 302 that turns the syringe pump controller 300 on and off, a power supply input 304 which electrically couples with the power supply 224, a pump connection port 306 that operatively couples with the pump 200, and an analog input 308 which couples with the pressure sensor 106 to receive the sensor signal 108. In this example, the sensor signal 108 is an analog signal which needs to be converted to digital signal via an analog-to-digital converter (ADC).

The syringe pump controller 300 also includes the display 222 that is implemented as a part of the syringe pump controller 300. The syringe pump controller 300 also includes a setpoint control knob 312 which allows the user to select which setpoint to use for the syringe pump controller 300 to operate during HFID procedures. Additionally, a reset switch 314 and a start switch 316 allows for the user to reset the setting and start the procedures, respectively. An emergency stop button 318 is also implemented to force stop the procedure by the user. In some examples, LED lights 310 can inform the user as to the current status of the controller 300 or the procedure, as well as informing the user if a device is not connected to the controller 300.

Listed below are some of the examples as to how the specific components of the syringe pump controller 300 is configured. For example, the analog input is 1 volt of direct current (VDC) equaling 100 mmHg of pressure, and the power supply 224 provides power of 24 VDC at 100 mA of current. For example, the pump 200 that is used is a model NE 8000 high pressure programmable single syringe pump as produced by New Era Pump Systems Inc. Although not shown in FIG. 3, the syringe pump controller 300 also includes an export port such as a USB port that is operatively coupled to the transmitter or output 218. In some examples, the USB port allows for a flash drive to be inserted such that the flash drive is capable of storing data as the pump 200 operates and sensor data is gathered. The setpoint control knob 312 may have any suitable number of increments ranging from 0 to 100 mmHg of pressure to be used as the setpoint for operation. For example, the reset switch 314 does not reset the processing unit 206 and only a power cycle is capable of resetting the processing unit 206.

Regarding the LED lights 310, as shown in FIG. 3, the “READY” LED is to indicate that all sub-processes are complete and the system is ready to start when the LED is turned on, and if blinking, a pump program has started and the pump 200 is in motion. The “OVER PRESSURE” LED indicates that the measured pressure has exceeded the setpoint and must be reset to clear error if it is blinking, and normal if turned off. The “PUMP COMMS” LED indicates that the communications have been established with the pump 200 if turned on, and if blinking indicates that the main computer 220 is currently trying to establish pump communication but has not succeeded. Most common problem would be a bad or missing connection to the pump 200 or if the pump 200 itself is turned off. The “FLASH DRIVE” LED indicates that the USB flash drive required for data storage and programming of the pump 200 is missing if blinking slowly, indicates that the USD flash drive was found but the pump program required for the pump 200 is missing if blinking fast, and indicates that the USB flash drive was found and the pump program is uploaded to the syringe pump if turned on solidly without blinking. The pump program provided by the USB flash drive can be customized to suit the need of each user.

The analog input 308 is a Bayonet Neill-Concelman (BNC) connector into which the catheter amplifier would be plugged and has a range between 0 to +1 VDC. The pump connection port 306 is an 8-pin circular connector containing two (2) different cables: a DB9 connector that plugs into the connector labeled TTL I/O, and an RJ-45 connector on a flat cable that plugs into the connector labeled RS-232. The power supply input 304 are two identical DC barrel connectors, wired in parallel which allows a single supply to power both the syringe pump controller 300 and the pump 200. Additionally, the syringe pump controller 300 may also have an Ethernet port that is a standard 8-pin RJ45 connector that allows for the user or a maintenance personnel of the device such as the system developer to gain access to the processing unit 206 located therein.

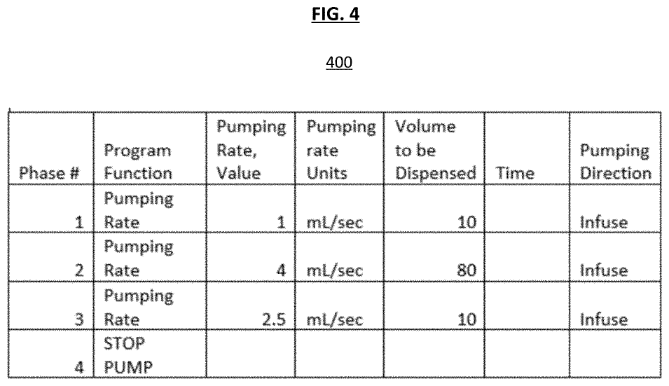

FIG. 4 shows an example of a pump program 400 which looks like a spreadsheet that indicates how to control how the pump 200 operates during the HIFD procedure. With the shown spreadsheet, the pump program 400 implements a pump profile that moves as follows: (1) 1 mL/sec for a volume of 10 mL; (2) 4 mL/sec for a volume of 80 mL; (3) 2.5 mL/sec for a volume of 10 mL; and (4) STOP PUMP. When finished, the pump 200 will have pushed a total of 100 mL over a period of 34 seconds. In some examples, the range of perfusion volumes is between 10 to 140 mL, the range of perfusion pressures is between 10 to 100 mmHg, and the prefusion rates ranges between 0.5 to 7 mL/sec. Other suitable ranges may also be implemented. After the spreadsheet is formed, the pump program 400 is saved in the USB flash drive to be inserted into the syringe pump controller 300, after which the controller 300 operates the pump 200 according to the pump program 400.

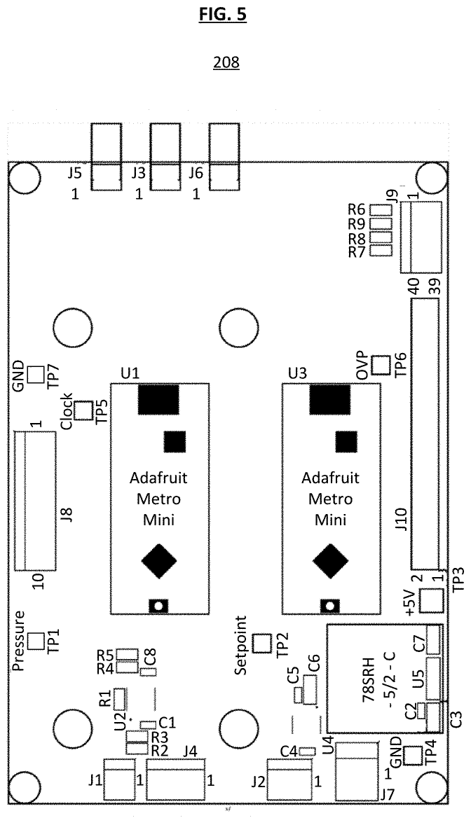

FIG. 5 shows an example of how the processing unit 206 can be implemented according to an embodiment. Specifically, the microcontroller 208 in the processing unit 206 is comprised of two Adafruit Metro mini ATmega328 based microcontrollers, each with a specific duty as follows: the U3 Metro Mini performs analog comparison of the pressure versus setpoint and shuts down the syringe pump if the pressure exceeds setpoint; and the U1 Metro Mini acts as the ADC to digitize the pressure and setpoint voltages, streams these values out the USB port to the main computer 220, and updates the display 222, or alternatively a user display such as the display of an external user device operatively coupled with the processing unit 206. Specifically, the pressure versus setpoint comparison is performed by an Arduino circuit on-board the U3 Metro Mini, which is performed using the pure analog comparator circuit, and the ADC circuit of the U1 Metro Mini converts the comparison result to digital data to export to the display and/or the main computer 220. The main computer 220 is a Raspberry Pi B3+(J10 in FIG. 5) whose functions are to: (1) upload the pump profile to the syringe pump from USB flash drive; (2) start the pump profile via RS-232 communicated pump command; (3) request current Volume Dispensed value via RS-232 pump command; and (4) store time stamped data to CSV file on the flash drive.

The analog signal from the catheter amplifier arrives at the BNC connector J11, continuing on to the single gain stage of U2A, amplifying the voltage to a maximum of +5 VDC. The signal then passes through a 1 kHz low pass, anti-aliasing filter. Diode D5 is a fast schottky with low forward voltage in place to protect the inputs of both U1 and U3 circuits. The design takes advantage of the analog comparator interrupt of the ATmega328 to produce very fast reactions with little to no jitter in the event of an over pressure event. The NE8000 syringe pump TTL I/O port, pin 2, is configured during the startup process on the Raspberry Pi B3+(J10). Pin 2 is configured to stop on high level, i.e. when the pressure reaches a setpoint as determined by the setpoint control knob 312.

The pump input is pulled high internally such that if the user neglects to connect the I/O cable, the pump will not start. Also, in various examples, the Raspberry Pi B3+(J10) is programmed to perform various ancillary tasks during all phases of the procedure, such as data storage, pump program loading, and pump start, but the Raspberry Pi B3+(J10) does not have the capability to stop the pump 200 because the process of stopping the pump 200 is under hardware control for safety reasons, e.g. to prevent user from inadvertently stopping the pump 200. As such, in such cases, the U1 Metro Mini and the U3 Metro Mini are responsible for stopping the pump 200 in case of an overpressure (i.e., the pressure exceeding the setpoint).

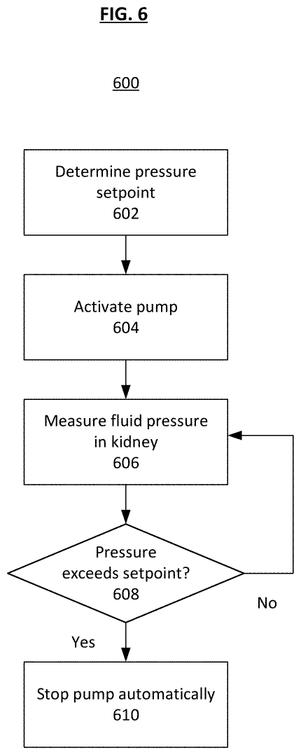

FIG. 6 shows a flow diagram of a method 600 by which the syringe pump controller 100 operates. In a first block 602, the syringe pump controller 100 determines the pressure setpoint as inputted by the user using the setpoint control knob 312. Then, in block 604, the pump 200 is activated by the syringe pump controller 100. As the pump 200 operates, the pressure sensor 106, operatively coupled with the syringe pump controller 100, measures the fluid pressure in the kidney in block 606 and relays the sensor signals 108 to the syringe pump controller 100. The syringe pump controller 100 then decides in block 608 if the pressure exceeds the setpoint as initially set in block 602. If the pressure does not exceed the setpoint, the process moves back to block 606 and the pressure sensor 106 continues measuring the fluid pressure. Otherwise, if the pressure exceeds the setpoint, the syringe pump controller 100 automatically stops the pump 200 in block 610 to prevent the kidney from suffering further damage. In the example of the syringe pump controller 300 using the microprocessor 208 as shown in FIG. 5, the maximum reaction time to the overpressure may be between 5 μsec and 7 μsec. In some examples, the block 610 occurs automatically upon detecting the pressure exceeding the setpoint, but in other examples, a warning LED light 310 may be turned on or a notification may appear on the display 222 requesting immediate user action.

Although the above examples describe administering saline solution to the patient, other solutions such as physiological solutions, physiological solutions containing therapeutic small molecules, and physiologic solutions containing gene expression vectors such as plasmids, siRNA, RNA, artificial chromosomes, or any other mode of gene transduction, transfection in mammalian cells/tissues, may also be injected using the catheter 102, the syringe 104, and the pump 200, which can be controlled by the syringe pump controller 100. Furthermore, macromolecules, proteins, protein-DNA or protein-RNA complexes and exosomes can also be delivered using the same. Additionally, stem cells or any other progenitor type cell line as well as organelles such as micronuclei and mitochondria can be delivered using the same.

The catheter 102, the syringe 104, the pump 200, and the syringe pump controller 100 can also be used in prevention of acute kidney injury, reversal of acute kidney injury, therapy of chronic kidney disease, therapy of genetic causes of kidney disease, therapy of hypertension, therapy of renal salt wasting syndromes, therapy of chronic interstitial nephritis, therapy of toxin induced kidney injury, and so on. Also, other types of hardware may be used for each of the aforementioned components. For example, the microcontroller 208 may be a central processing unit (CPU), a system-on-a-chip (SoC), or other types of integrated circuits that has data processing capabilities. The output 218 may be a wireless transmitter or other types of wired connector system such as USB-C.

While the present disclosure is amenable to various modifications and alternative forms, specific embodiments have been shown by way of example in the drawings and are described in detail below. The intention, however, is not to limit the present disclosure to the particular embodiments described. On the contrary, the present disclosure is intended to cover all modifications, equivalents, and alternatives falling within the scope of the present disclosure as defined by the appended claims.

What is claimed is:

1. A syringe pump controller comprising:
a processing unit comprising at least one microcontroller and a receiver, the microcontroller operatively coupled to a syringe pump and the receiver operatively coupled to a pressure sensor, the processing unit configured to:
receive pressure sensor data via the receiver,
determine whether the pressure sensor data exceeds a predetermined setpoint pressure, and
stop the syringe pump in response to determining that the pressure sensor data exceeds the setpoint pressure.

2. The syringe pump controller of claim 1, further comprising a catheter fluidly coupled with the syringe pump and configured to deliver fluid from the syringe pump.

3. The syringe pump controller of claim 1, wherein the pressure sensor is positioned parallel to the syringe pump.

4. The syringe pump controller of claim 1, wherein the pressure sensor is configured to measure a pressure within the catheter located distally of an exit portion of the syringe pump.

5. The syringe pump controller of claim 2, wherein the catheter comprises a first end fluidly coupled with the exit portion of the syringe pump and a second end where fluid delivered from the syringe pump exits.

6. The syringe pump controller of claim 5, wherein the pressure sensor is positioned at a body portion of the syringe pump.

7. The syringe pump controller of claim 5, wherein the pressure sensor is positioned at the first end of the catheter.

8. The syringe pump controller of claim 5, wherein the pressure sensor is positioned between the first end and the second end of the catheter.

9. The syringe pump controller of claim 2, further comprising a plurality of syringe pumps each configured to deliver a predetermined fluid at a predetermined rate into the catheter.

10. The syringe pump controller of claim 9, wherein each of the plurality of syringe pumps comprises an exit portion fluidly coupled with the first end of the catheter.

11. The syringe pump controller of claim 9, wherein the processing unit is configured to stop one or more of the plurality of syringe pumps in response to determining that the pressure sensor data in the catheter exceeds the setpoint pressure.

12. A method of operating a syringe pump, comprising:
determining, by a processing unit operatively coupled with the syringe pump, a pressure setpoint;
activating, by the processing unit, the syringe pump;
measuring, by a pressure sensor, fluid pressure;
determining, by the processing unit based on the fluid pressure, whether the measured fluid pressure exceeds the pressure setpoint; and
in response to determining that the measured fluid pressure exceeds the pressure setpoint, automatically stopping, by the processing unit, the syringe pump.

13. The method of claim 12, wherein the pressure sensor is positioned at the syringe pump or a catheter extending from the syringe pump.

14. The method of claim 12, further comprising a plurality of syringe pumps configured to be operated by the processing unit.

15. The method of claim 14, wherein the processing unit is configured to stop one or more of the plurality of syringe pumps in response to determining that the pressure sensor data in the catheter exceeds the setpoint pressure.
